(19) United States
(12) Patent Application Publication
(10) Pub. No.: US 2009/0192075 A1
(43) Pub. Date: Jul. 30, 2009

(54) Amylin Formulations

(75) Inventor: Solomon S. Steiner, Mount Kisco, NY (US)

Correspondence Address: Pabst Patent Group LLP, 1545 PEACHTREE STREET NE, SUITE 320, ATLANTA, GA, 30309, US

(73) Assignee: Biodel Inc.

(21) Appl. No.: 12/410,754

(22) Filed: Mar. 25, 2009

Related U.S. Application Data
(63) Continuation of application No. 12/062,355, filed on Apr. 3, 2008.
Application No. 12/062,355 is a continuation-in-part of application No. 11/767,698, filed on Jun. 25, 2007.
Application No. 11/767,698 is a continuation of application No. 11/077,604, filed on Mar. 11, 2005, now Pat. No. 7,279,457.
(60) Provisional application No. 60/910,036, filed on Apr. 4, 2007.
(60) Provisional application No. 60/990,811, filed on Nov. 28, 2007.
(60) Provisional application No. 60/552,637, filed on Mar. 12, 2004.
(60) Provisional application No. 60/609,194, filed on Sep. 9, 2004.

(51) Int. Cl. A61K 38/28; A61P 3/10

(52) U.S. Cl. 514/4

(57) ABSTRACT

A combined insulin and amylin and/or GLP-1 mimetic formulation has been developed wherein the pH of the insulin is decreased so that the amylin and/or GLP-1 remains soluble when mixed together with the insulin. In the preferred embodiment, a bolus insulin is administered by injection before breakfast, providing adequate bolus insulin levels to cover the meal, without producing hypoglycemia after the meal. This can be combined with an adequate basal insulin for 24 hours. Lunch and dinner can be covered by two bolus injections of the insulin and amylin and/or GLP-1 mimetic combination. A GLP-1 mimetic may be combined with either rapid acting or basal insulin formulations. As a result, a patient using the combination formulation therapy may only need to inject half as many times per day.

### PRIORITY

This application claims priority to U.S. Ser. No. 60/910,036 filed on Apr. 4, 2007, and U.S. Ser. No. 60/990,811 on Nov. 28, 2007.

### FIELD OF THE INVENTION

The present invention generally relates to formulations combining amylin or other adjunct components forming aggregates and insulin in solution.

### BACKGROUND OF THE INVENTION

Intensive insulin therapy for diabetes involves providing a basal insulin, ideally present at a uniform level in the blood over a 24 hour period, and a bolus or meal time (prandial) insulin to cover the added carbohydrate load from digestion concomitant with each meal.

In 1936, Hans Christian Hagedorn and B. Norman Jensen discovered that the effects of injected insulin could be prolonged by the addition of protamine obtained from the “milt” or semen of river trout. The insulin was added to the protamine and the solution was brought to pH 7 for injection. In 1946, Nordisk Company was able to form crystals of protamine and insulin and marketed it in 1950 as NPH, (Neutral Protamine Hagedorn, “NPH”) insulin. NPH insulin has the advantage that it can be mixed with an insulin that has a faster onset to compliment its longer lasting action. Eventually all animal insulins were replaced by human recombinant insulin.

Until very recently, basal insulin was usually provided by the administration of two daily doses of NPH insulin, separated by 12 hours. A patient eating three meals a day and using NPH insulin as the basal insulin required five injections per day, one with each of three meals and two NPH insulin injections, one in the morning and the other at bedtime. To reduce the number of injections the patient had to take, the morning dose of NPH insulin was combined with a short acting insulin, (recombinant human insulin) or a rapid acting insulin analog, such as lispro. A typical combination was a 70% NPH to 30% rapid acting insulin analog mixture. As a result, the patient could reduce the number of injections from five per day to four per day. See, for example, Garber, Drugs 66(1):31-49 (2006).

More recently insulin glargine, (trade name LANTUS®) a “very long-acting” insulin analog, has become available. It starts to lower blood glucose about one hour after injection and keeps working evenly for 24 hours. J. Rosenstock and colleagues found that patients who took insulin glargine had a much lower risk of low blood glucose (hypoglycemia) than the patients who took NPH insulin.

Amylin (islet amyloid polypeptide) is a hormone with suggested roles in the regulation of glucose homeostasis, gastric motor and secretory function and gastroprotection. Amylin is believed to aid in limiting glycemic excursions by slowing gastric emptying, promoting satiety, and inhibiting inappropriate secretion of glucagon, a catabolic hormone that opposes the effects of insulin and amylin. SYMLIN™ (pramlintide acetate) is a synthetic analog of Amylin, It is commercially available for injection, and may not be mixed with insulin, glucose control during the postprandial period.

Another injectable polypeptide intended for diabetics is glucagon like peptide hormones (GLP-1). This hormone is normally produced in the GI tract and in some regions of the brain. This hormone influences the response of beta cells to glucose and operated in concert with other islet hormones to limit glucose excursions. An analog of Glp-1 comercially known as “BYETTA™”(exenatide) is in a class of medicines for type 2 diabetes called incretin mimetics.

Unfortunately, pramlintide and exenatide cannot be mixed with most forms of insulin because the mixture causes aggregates to form and precipitate prior to injection. Administration of a precipitated insulin makes it virtually impossible to administer a known and reliable dose.

There is a need for formulations containing amylin and insulin which does not aggregate and which can be administered for the treatment of diabetes.

It is therefore an object of the present invention to provide formulations of insulin in combination with pramlintide and/or exenatide or a variant thereof.

It is another object of the present invention to provide a basal-bolus insulin and amylin combination formulation.

### SUMMARY OF THE INVENTION

A combined insulin and amylin analog such as pramlintide or incretin mimetic or GLP-1 mimetic such as exenatide formulation has been developed wherein the pH of the insulin is decreased so that the amylin remains soluble when mixed with the insulin.

In one embodiment, a rapid acting insulin and amylin formulation is administered via subcutaneous injection before breakfast, providing adequate insulin levels to cover the meal. In another embodiment, a rapid acting insulin, basal insulin and pramlintide (SYMLIN®) are combined to form a clear solution, to provide adequate basal insulin for up to 24 hours. Lunch and dinner can be covered by two bolus injections of the combined insulin and amylin formulation. As a result, a patient using the combination formulation may only have to inject half as many times per day as is typical. In another embodiment insulin is combined with enenatide, which may also reduce the number of injections needed per day.

### DETAILED DESCRIPTION OF THE INVENTION

I. Compositions

In one embodiment, the composition contains insulin or a biologically active variant or fragment thereof in combination with amylin or a biologically active variant or fragment thereof or an “incretin mimetic” or GLP-1 mimetic (collectively referred to as adjunct compounds). The insulin is provided at a low pH, at which the amylin does not precipitate or aggregate when mixed together, even over a wide range of ratios of rapid acting to long acting insulin.

The literature has reported that studies of amylin₂₀₋₂₉ and several variant peptides reveal that low levels of deamidation can have a significant effect on the secondary structure and aggregation behavior of these molecules. The peptide can spontaneously deamidate, and the presence of less than 5% of deamidation impurities leads to the formation of aggregates that have the hallmarks of amyloid. Deamidation leads to an unexpected pH dependence of the aggregation behavior, and furthermore, trace amounts of deamidated material can induce aggregation when added to freshly purified samples. In the absence of impurities the peptide does not spontaneously aggregate and is not amyloidogenic (Raleigh et al., Protein Science, 11: 342-349 (2002)). Without being bound by any one theory, it is possible that the presence of insulin and/or adjusting the pH of insulin inhibits the deamidation of amylin and thus prevents the formation of aggregates.

Definitions

Amylin (Islet Amyloid Polypeptide) or IAPP is a 37 amino acid peptide secreted by the pancreatic B cells at the same time as insulin. It is a natural adjunct to insulin and serves as a synergistic partner in glycemic control (Insulin/Amylin ratio=100:1). Analogs of amylin are known and described in the art. Amylin analogs include not only natural occurring forms of amylin, (including human, pig, sheep, or other animal source), but also proteins which have an amino acid sequence substantially similar to that of amylin but containing one or more deletions or additions of amino acids within that sequence or including substitution of one or more amino acids, generally with a conserved amino acid, for example, glycine may be replaced with a valine, or a positively charged amino acid replaced with other positively charged amino acid, and having similar or identical biological activity to the naturally occurring amylin most closely related in sequence to that analog. Representative amylin analogs including AC-0137, a human amylin having the amidated amino acid sequence between amino acids 30 and 37, PRAMLINTIDE™, which delays gastric emptying in type I diabetics, and SYMLIN®,having two amino acid substitutions compared to native amylin. It is positively charged and delivered as an acetate salt. Unless otherwise stated, reference to “amylin” including amylin and amylin analogs having similar function and structure.

As used herein, “insulin” refers to human or non-human, recombinant, purified or synthetic insulin or insulin analogues having similar function and structure, unless otherwise specified.

As used herein, “Human insulin” is the human peptide hormone secreted by the pancreas, whether isolated from a natural source or made by genetically altered microorganisms. As used herein, “non-human insulin” from an animal source such as pig or cow.

As used herein, an insulin analogue is an altered insulin, different from the insulin secreted by the pancreas, but still available to the body for performing the same action as natural insulin. Through genetic engineering of the underlying DNA, the amino acid sequence of insulin can be changed to alter its ADME (absorption, distribution, metabolism, and excretion) characteristics. Examples include insulin lispro, insulin glargine, insulin aspart, insulin glulisine, insulin detemir. The insulin can also be modified chemically, for example, by acetylation. As used herein, human, insulin analogues are altered human insulin which is able to perform the same action as human insulin.

As used herein, a “chelator” or “chelating agent”, refers to a chemical compound that has the ability to form one or more bonds to zinc ions. The bonds are typically ionic or coordination bonds. The chelator can be an inorganic or an organic compound. A chelate complex is a complex in which the metal ion is bound to two or more atoms of the chelating agent.

As used herein “incretin mimetic” is a distinct class of agents used to treat diabetes. An incretin mimetic works to mimic the anti-diabetic or glucose-lowering actions of naturally occurring human hormones called incretins. These actions include stimulating the body's ability to produce insulin in response to elevated levels of blood sugar, inhibiting the release of a hormone called glucagon following meals, slowing the rate at which nutrients are absorbed into the bloodstream and reducing food intake.

As used herein, “GLP-1 mimetics” are, as their name implies, substances that mimic the effects of incretin hormones but are not as vulnerable to the actions of DPP-IV as GLP-1. Incretin mimetics work as receptor agonists. The primary GLP-1 mimetic is exenatide (synthetic exendin-4), which is now approved in the United States.

As used herein, a “solubilizing agent”, is a compound that increases the solubility of materials in a solvent, for example, insulin in an aqueous solution. Examples of solubilizing agents include surfactants (TWEEN®); solvent, such as ethanol; micelle forming compounds, such as oxyethylene monostearate; and pH-modifying agents.

As used herein, a “dissolution agent” is an acid that, when added to insulin and EDTA, enhances the transport and absorption of insulin relative to HCl and EDTA at the same pH, as measured using the epithelial cell transwell plate assay described in the examples below. HCl is not a dissolution agent but may be a solubilizing agent. Citric acid is a dissolution agent when measured in this assay.

As used herein, an “excipient” is an inactive substance other than a chelator or dissolution agent, used as a carrier for the insulin or used to aid the process by which a product is manufactured. In such cases, the active substance is dissolved or mixed with an excipient.

As generally used herein, a drug is considered “highly soluble” when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water. A drug is considered highly soluble when 90% or more of an administered dose, based on a mass determination or in comparison to an intravenous reference dose, is dissolved. Solubility can be measured by the shake-flask or titration method or analysis by a validated stability-indicating assay.

As generally used herein, an immediate release drug formulation is considered “rapidly dissolving” when no less than 85% of the labeled amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

A. Insulin

There are several differing types of commercial insulin available for diabetes patients. These types of insulins vary according to (1) how long they take to reach the bloodstream and start reducing blood glucose levels; (2) how long the insulin operates at maximum strength; and (3) how long the insulin continues to have an effect on blood sugar.

Rapid and Intermediate Acting Insulin.

Some diabetes patients use rapid-acting insulin at mealtimes, and also long-acting insulin for ‘background’ continuous insulin. This type of insulin starts working within 6 hours of administration and provides a continuous level of insulin activity for up to 36 hours. Long-acting insulin operates at maximum strength after about 8-12 hours, sometimes longer.

At present there are three types of rapid-acting commercial insulin available: Humalog® (Lispro® or Lysine-Proline insulin), Apidra®, and ASPART insulin. Biodel also has a proprietary insulin formulation that is in clinical trials, referred to as VIAJECT™. This is an insulin formulated with EDTA and citric acid, having a pH of 4.0.

Characterized by a cloudy appearance, intermediate-acting insulin has a longer lifespan than short-acting insulin but it is slower to start working and takes longer to reach its maximum strength. Intermediate-acting insulin usually starts working within 2-4 hours after injection, peaks somewhere between 4-14hours and remains working for approximately 24 hours.

Types of intermediate-acting insulin include NPH (Neutral Protamine Hagedorn) and LENTE insulin. NPH insulin contains protamine which slows down the speed of absorption so that the insulin takes longer to reach the bloodstream but has a longer peak and lifespan. This means that fewer insulin injections are needed each day.

Long Acting Insulin

LANTUS™ (glargine) is a recombinant human insulin analog that can have up to a 24 hour duration. It differs from human insulin by having a glycine instead of asparagine at position 21 and two arginines added to the carboxy-terminus of the beta-chain. LANTUS™ consists of insulin glargine dissolved in a clear aqueous fluid (100 IU, 3.6378 mg insulin glargine, 30 micrograms zinc, 2.7 mg m-cresol, 20 mg glycerol 85%, and water to 1 ml). The pH is adjusted with HCl to 4.0.

The median time between injection and the end of pharmacological effect was 14.5 hours (range 9.5 to 19.3 hours) for NPH human insulin, and 24 hours (range 10.8 to greater than 24.0 hours) for insulin glargine.

The package insert says not to mix LANTUS™ with any other types of insulin, unlike most rapid acting and intermediate acting insulins.

VIAJECT™

Formulations include insulin, a chelator and a dissolution agent(s) and, one or more other excipients as required to make a formulation suitable for subcutaneous administration. The choice of dissolution agent and chelator, the concentration of both the dissolution agent and the chelator, and the pH that the formulation is adjusted to, all have a profound effect on the efficacy of the system. While many combinations have efficacy, the preferred embodiment is chosen for many reasons, including safety, stability, regulatory profile, and performance.

In the preferred embodiment, at least one of the formulation ingredients is selected to mask any charges on the active agent. This may facilitate the transmembrane transport of the insulin and thereby increase both the onset of action and bioavailability for the insulin. The ingredients are also selected to form compositions that dissolve rapidly in aqueous medium. Preferably the insulin is absorbed and transported to the plasma quickly, resulting in a rapid onset of action (preferably beginning within about 5 minutes following administration and peaking at about 15-30 minutes following administration).

The chelator, such as EDTA, chelates the zinc in the insulin, thereby removing the zinc from the insulin solution. This shifts the equilibrium toward the dimeric and monomeric form and retards reassembly into the hexamer state. Since these two forms exist in a concentration-driven equilibrium, as the monomers are absorbed, more monomers are created. Thus, as insulin monomers are absorbed, additional dimers dissemble to form more monomers. The monomeric form has a molecular weight that is less than one-sixth the molecular weight of the hexameric form, thereby markedly increasing both the speed and quantity of insulin absorbed. To the extent that the chelator (such as EDTA) and/or dissolution agent (such as citric acid) hydrogen bond with the insulin, it is believed that it masks the charge on the insulin, facilitating its transmembrane transport and thereby increasing both the onset of action and bioavailability for insulin.

The insulin can be recombinant or purified from a natural source. The insulin can be human or non-human. Human is preferred. In the most preferred embodiment, the insulin is human recombinant insulin. Recombinant human insulin is available from a number of sources. The insulin may also be an insulin analogue which may be based on the amino acid sequence of human insulin but having one or more amino acids differences, or a chemically modified insulin or insulin analog.

The dosage of the rapid acting or insulin depends on the patient to be treated. Insulin is generally included in a dosage range from 3 to 100 IU.

Certain acids appear to mask charges on the insulin, enhancing uptake and transport. Those acids which are effective as dissolution agents include acetic acid, ascorbic acid, citric acid, glutamic, aspartic, succinic, fumaric, maleic, and adipic. For example, if the active agent is insulin, a preferred dissolution agent is citric acid. The hydrochloric acid may be used for pH adjustment, in combination with any of the formulations, but is not a dissolution agent.

In the preferred embodiment, the rapid acting insulin has a zinc chelator mixed with the active agent. The chelator may be ionic or non-ionic. Suitable chelators include ethylenediaminetetraacetic acid (EDTA), ethylene-bis(oxyethylene nitro) tetraacetic acid (EGTA), di-, tri-sodium citrate, chlorella, cilantro, 1,2,-Diaminocyclohexanetetraacetic acid (CDTA), dimercaptosuccinic acid (DMSA). Hydrochloric acid is used in conjunction with TSC to adjust the pH, and in the process gives rise to the formation of citric acid, which is a dissolution agent.

In the preferred embodiment, the chelator is EDTA. For example, when the active agent is insulin, it is known that the chelator captures the zinc from the insulin, thereby favoring the dimeric form of the insulin over the hexameric form and facilitating absorption of the insulin by the tissues surrounding the site of administration (e.g. mucosa, or fatty tissue). In addition, the chelator hydrogen may bond to the active agent, thereby aiding the charge masking of the active agent and facilitating transmembrane transport of the active agent.

The range of chelator corresponds to an effective amount of EDTA in combination with insulin and citric acid of between 2.42×10⁻⁴ M to 9.68×10⁻² M EDTA.

B. Amylin

Amylin is a peptide involved in maintaining glucose homeostais. It is found in beta cells of pancreas and to some extent in gastrointestinal tract and nervous system. Amylin works along with insulin to regulate blood glucose levels by suppressing secretion of glucagons after eating and restraining the rate at which stomach is emptied. Two amylin fragments identified in vivo are: aa24-37 of human amylin and aa17-37 of human amylin. Amylin, similar to insulin, is absent or deficient in patients with diabetes. When used with insulin, this compound can help patients achieve improved glycemic control with additional benefits that cannot be realized with insulin alone.

The amino acid sequence of amylin (SEQ ID NO:1) is:

### KCNTATCATQRLANFLVHSSNNFGAILSSTNVGSNTY-(NH2)

Pramlintide acetate (SYMLIN™) is an adjunct treatment for diabetes (both type 1 and 2). It is derived from amylin, a hormone that is released into the bloodstream, in a similar pattern as insulin, after a meal.

The amino acid sequence for Pramlintide (SEQ ID NO:2) is:

### KCNTATCATNRLANFLVHSSNNFGPILPPTNVGSNTY-(NH2)

By substituting for endogenous amylin, pramlintide aids in the absorption of glucose by slowing gastric emptying, promoting satiety, and inhibiting inappropriate secretion of glucagon, a catabolic hormone that opposes the effects of insulin and amylin.

Symlin™ has been approved for use by the FDA by type 1 and type 2diabetics who use insulin. Symlin™results in weight loss, allows patients to use less insulin, lowers average blood sugar levels, and substantially reduces what otherwise would be a large unhealthy rise in blood sugar that occurs in diabetics right after eating. Symlin™ is the only drug approved by the FDA to lower blood sugar in type 1 diabetics since insulin's discovery in the early 1920s.

C. GLP-1

GLP-1 is an incretin that stimulates insulin secretion. It is being used along with other therapies, for patients with Type 2 Diabetes Mellitus (TTDM). It is a gut hormone released by the L cells in the lower small intestine and is triggered by nutrients and may also stimulate islet proliferation and islet cell neogenesis. GLP-1 inhibits glucagon secretion, gastric emptying and food intake.

GLP-1 requires continuous subcutaneous infusion due to its half life. The natural GLP-1 has a half life of 1-2 minutes in humans, and therefore is currently not a practical therapy for TTDM. Therefore, incretin mimetics have been used to bind to the dgradd amided, GLP-1. Exenatide is one of these incretin mimetics, commercially known as Byetta™, which has an increased half-life due to the alteration of the amide that prolongs DPP-4 cleavage. Exenatide or other altered GLP-1 amides has a longer half life, approximately 2.4 hours, however it still must be injected twice daily in order to be used as a therapy for TTDM.

D. Excipients

Insulin is combined with amylin or a biologically active fragment thereof at a pH at which the amylin is soluble, typically 4.0. In one embodiment, there is no precipitate formed on mixing amylin with Viaject™ which has a pH of 4. Ultimately, this combination provides insulin and amylin to shut down hepatic gluconeogenesis, carry the patient through a meal with less bolus insulin, thereby reducing the chance of hypoglycemia and provides 24 hr long lasting basal insulin, reducing the number of injections required/day from four to three.

pH is typically adjusted with food acids and bases (e.g. sodium bicarbonate), and alcohols, and buffer salts for pH control. Suitable dissolution agents include citric acid and hydrochloric acid. A preferred dissolution agent is citric acid.

The formulation may also include a metal chelator. The chelator may be ionic or non-ionic. Suitable chelators include ethylenediaminetetraacetic acid (EDTA), citric acid, dimercaprol (BAL), penicillamine, alginic acid, chlorella, cilantro, alpha lipoic acid, dimercaptosuccinic acid (DMSA), dimercaptopropane sulfonate (DMPS), and oxalic acid. In the preferred embodiment, the chelator is EDTA. In addition to charge masking, it is believed that the chelator pulls the zinc away from the insulin, thereby favoring the monomeric form of the insulin over the hexameric form and facilitating absorption of the insulin by the tissues surrounding the site of administration (e.g. mucosa, or fatty tissue). Optionally, the chelator and solubilizing agent are the same compound. Ions may be part of the active agent, added to the stabilizing agent, mixed with the chelator, and/or included in the coating. Representative ions include zinc, calcium, iron, manganese, magnesium, aluminum, cobalt, copper, or any divalent metal or transitional metal ion. Zn⁺² has a stronger binding affinity for EDTA than Ca⁺².

II. Methods of Administration

The combination formulation of insulin and amylin usually is given by subcutaneous (beneath the skin) injection. Insulin is generally included in a dosage range of 3-100 IU per human dose. SYMLIN®, an adjunct treatment for diabetes (both type 1 and 2) derived from amylin is administered in 15, 30, 45,60, and 120 mcg doses according to the package insert.

The amount of insulin needed depends on diet, other diseases, exercise, and other drugs and may change with time. A doctor can determine how often and at what time of day to inject the insulin, as well as what type of insulin will best control the level of sugar in the blood.

The different types of insulin vary as to how quickly they start to work and how long they go on reducing the amount of blood sugar. For example, rapid-acting insulins, such as regular insulin and Semilente, start to work in 30-60 minutes and go on working for 5-16 hours; long-acting insulins, such as Ultralente, start to work in 4-8 hours and continue working for 36 hours. The combination formulation can be adjusted to provide for continuous results over an extended period of time, with resulting schedules requiring injections once, twice or three times a day.

The formulation is designed to be rapidly absorbed and transported to the plasma for systemic delivery. Formulations may be administered to a type 1 or type 2 diabetic patient before or during a meal. The formulation is typically administered by subcutaneous injection. Due to the rapid absorption, the compositions can shut off the conversion of glycogen to glucose in the liver, thereby preventing hyperglycemia, the main cause of complications from diabetes and the first symptom of type 2 diabetes.

The present invention will be further understood by reference to the following non-limiting example.

### EXAMPLE 1

### VIAjec™ can be Mixed with Symlin®, Eliminating the Need for a Separate Injection.

Materials and Methods

VIAjec™ insulin was mixed in a ratio of 25U VIAject™ to 120 μg SYMLIN® to simulate a high dose of both.

The individual solutions were visually inspected prior to and after mixing together in a clear sterile vial. Each individual solution was molecularly sized using a Malvern Zetasizer. The original and combined materials were assayed by HPLC for insulin and pramlintide content respectively.

Materials were stored at 4° C. and reanalyzed using the same methods on day 1 and 4 post mixing.

Results

Visual inspection showed that the mixed solution of SYMLIN® and VIAject™ were clear after initial mixing and remained clear after one hour at room temperature.

Further visual inspection on days 1, 4, 7 and 11 after mixing showed that the combined product remained clear. These observations were confirmed by molecular sizing using a Malvern Zetasizer. Using HPLC analysis, the two peaks of insulin and Symlin retained their full area counts throughout the 11 day study period. These results show that these two formulations appear stable by HPLC analysis, since there was little change in the peak areas, and the percent insulin a-21 desamido did not increase (Table 1).

TABLE 1
HPLC analysis:
(10 μl injection, insulin assay, 210 nm).
Chromatograms showed well separated peaks.

| | Viaject area (RT 6.9 m) | % | Symlin area (4.35 m) | % |
|---|---|---|---|---|
| Day 1 | 25,485,096 | 87.5 | 3,341,918 | 11.47 |
| Day 4 | 25,544,486 | 87.4 | 3,348,759 | 11.42 |
| Day 7 | 25,738,554 | 87.3 | 3,424,829 | 11.62 |
| Day 11 | 25,838,934 | 87.5 | 3,467,738 | 11.7 |

| | | | | |
|---|---|---|---|---|
| Peak Name | RT | Area | % Area | Height |

Symlin ® and VIAject ™ Mixture at 1 Day:

| | | | | |
|---|---|---|---|---|
| Symlin | 4.436 | 3341918 | 11.47 | 244663 |
| Insulin | 7.109 | 25485096 | 87.49 | 1111926 |
| A-21 desamido | 9.005 | 303752 | 1.04 | 12567 |

Symlin ® and VIAject ™ Mixture at Day 4:

| | | | | |
|---|---|---|---|---|
| Symlin | 4.610 | 3348759 | 11.46 | 235991 |
| Insulin | 7.597 | 25544486 | 87.44 | 1040070 |
| A-21 desamido | 9.698 | 320178 | 1.10 | 11614 |

Symlin ® and VIAject ™ Mixture at Day 11:

| | | | | |
|---|---|---|---|---|
| Symlin | 5.147 | 3467738 | 11.70 | 216341 |
| Insulin | 9.141 | 25838934 | 87.15 | 887021 |
| A-21 desamido | 11.877 | 342655 | 1.16 | 11292 |

1. A composition comprising insulin, a zinc chelator, and a dissolution agent in combination with one or more adjunct compounds selected from the group consisting of amylin and GLP-1 mimics or analogs thereof, wherein the pH of the composition is adjusted to solubilize the adjunct compound and prevent aggregation.

2. The composition of claim 1, comprising rapid acting insulin.

3. The composition of claim 1, comprising intermediate acting insulin.

4. The composition of claim 1, having a pH of 4.0.

5. The composition of claim 1, wherein the chelator is selected from the group consisting of ethylenediaminetetraacetic acid (EDTA), ethylene-bis(oxyethylene nitro) tetraacetic acid (EGTA), trisodium citrate (TSC), alginic acid, alpha lipoic acid, dimercaptosuccinic acid (DMSA), 1,2,-diaminocyclohexanetetraacetic acid (CDTA).

6. The composition of claim 5, wherein the chelator is ethylenediaminetetraacetic acid (EDTA).

7. The composition of claim 1, wherein the dissolution agent is an acid selected from the group consisting of acetic acid, ascorbic acid, citric acid, glutamic acid, succinic acid, aspartic acid, maleic acid, fumaric acid, and adipic acid.

8. The composition of claim 1, comprising amylin or an amylin analog.

9. The composition of claim 1, comprising GLP-1 or a mimic thereof.

10. The composition of claim 1, comprising rapid acting insulin, citric acid, and EDTA.

11. A method of treating an individual in need thereof with insulin comprising administering to the individual a composition comprising insulin, a zinc chelator, and a dissolution agent in combination with one or more adjunct compounds selected from the group consisting of amylin and GLP-1 mimics or analogs thereof, wherein the pH of the composition is adjusted to solubilize the adjunct compound and prevent aggregation.

12. The method of claim 11 wherein the composition is administered by injection.

13. The method of claim 11 wherein a bolus insulin is administered by injection before breakfast.

14. The method of claim 13 further comprising administering basal insulin in an amount effective for 24 hours.

15. The method of claim 11 comprising administering two bolus injections of the insulin and adjunct formulation.

16. The method of claim 11 comprising providing a GLP-1 mimetic combined with either rapid acting or basal insulin formulations.


SEQUENCE LISTING (2 sequences)

SEQ ID NO: 1 - protein, 37 aa, Homo sapiens [sequence omitted]

SEQ ID NO: 2 - protein, 37 aa, Artificial Sequence
  Amylin derivative
KCNTATCATNRLANFLVHSSNNFGPILPPTNVGSNTY